(19) United States
(12) Patent Application Publication
(10) Pub. No.: US 2019/0108264 A1
(43) Pub. Date: Apr. 11, 2019

(54) SYSTEM PROTOTYPE OF INTELLIGENT REGIONAL MOBILE HEALTHCARE INTEGRATED DATA CENTER BASED ON METADATA

(71) Applicant: Shanghai DataCenter Science Co., LTD, Shanghai (CN)

(72) Inventors: Jun ZHANG, Shanghai (CN); Yong Fang, Wuxi City (CN); Bin Wang, Shanghai (CN)

(21) Appl. No.: 15/984,520

(22) Filed: May 21, 2018

(30) Foreign Application Priority Data
Oct. 9, 2017 (CN) 201710928036.X

(51) Int. Cl. G06F 17/30; G16H 10/60

(52) U.S. Cl. CPC G06F 17/30563; G16H 10/60

(57) ABSTRACT

The present invention discloses a system prototype of an intelligent regional mobile healthcare integrated data center based on metadata, characterized by comprising the following steps: collecting data from a mobile medical aid post, extracting and purifying the data, and loading same in a regional mobile healthcare integrated data center; providing the data for a data warehouse by aggregating the data; according to categories of mobile healthcare, extracting corresponding professional metadata from the data warehouse, to form a data market; and mapping metadata of a knowledge base to dictionary data of a service system by matching and merging logics, to provide uniform analysis paths and data services. The system prototype of an intelligent regional mobile healthcare integrated data center based on metadata is constructed.

### TECHNICAL FIELD

The present invention relates to a system prototype of an intelligent regional mobile healthcare integrated data center based on metadata,

### BACKGROUND

With the setup of a regional intelligent healthcare serving platform, intelligent healthcare will really implements interconnection and interworking of healthcare information. Intelligent healthcare engineering will be a multifaceted data processing platform, which implements fusion of intelligent healthcare information finally by correlating, estimating and combining data of a plurality of information sources and fully processing and cooperatively using associated information about various systems and multivariate data of the Internet of things.

The process of forming the industrial structure and profit chain of intelligent healthcare is complicated, so that new commercial modes and serving methods are needed to integrate profit relationships, adjust the profit structure and change the allocation manner. So far, the technology, standard, product, application and policy of an intelligent healthcare system respectively have made a good start.

The intelligent healthcare system relates to a plurality of links such as information collection, network communication, information processing, terminal interface, functional structure and the like. The existing services and technical standards relate to a wide range of field, various standards, which are intertwined, disordered, and complicated and confused, are formed, and a large number of standards are out of date and are required to be reestablished. For a long time, medical institutions have formed a pattern of solidification. Network integration and service provider structures are complicated. There is no effective means for resource allocation manner, technical coordination and docking, service coordination management and the like, and there is no effective constraint and management for market, and technical resource allocation.

According to the service field of hospitals, integrated data centers of hospitals may be divided into data models of the field mainly storing clinical services, the field of data models thereof may be divided into patient management field, doctor's advice field, laboratory field, observation field, case field, case history field, surgical operation field, nursing field and the like; and dictionary data stored in systems mainly storing hospital services, and national standards, professional standards, local standards, hospital departments, service units, service personnel, service items and other user-defined metadata, which are used in the hospital services.

A data warehouse is a significant data basis providing support for statistical analysis of hospitals. A data source of the data warehouse is obtained by calculating and aggregating data of the integrated data center of a hospital. Meanwhile, the data warehouse also provides a data source for establishment of a data market.

The data market is a warehouse of collecting data from a piece of operated data and other data source serving a certain special professional group. In terms of scope, data is extracted from a hospital database, a data warehouse or a more professional data warehouse.

In order to support the standardization of hospital diagnosis and treatment services and provide a correct guide for operations of medical staff, the platform should possess an independent knowledge base. Data of the knowledge base may come from guides, specifications, literatures, management rules and the like which are related to healthcare and achieve the effect of assisting medical staff through the flow of embedding the services of the platform in the hospital service system.

In a hospital information system, a mobile healthcare subsystem is a very special healthcare information system, has high requirements for mobility and timeliness of the system, especially in healthcare of wound, cardiovascular disease and diabetes, there is a need to enhance the intellectualization of a mobile healthcare system because time is life; and is integrated with the whole hospital information system, to provide mobile healthcare services for patients within a certain range; and is cleaned first to form a data warehouse, then is classified to form a data supermarket, and is labeled finally to form a knowledge base.

Metadata is a statement made on a certain potential informational object. A metadata pattern refers to a rule set, which stipulates what types of subject-verb-object statements are allowed and how to make such statements. In the metadata pattern, one element is a statement made on a certain resource, and meanwhile, may be used for nominating a certain attribute of the resource. A value is data allocated to a certain element.

There are a variety of correlations among mobile healthcare. One mobile healthcare may be divided into a plurality of knowledge units. There are correlations between examination indexes and symptoms of different mobile healthcare, and there are correlations among various treatment means. Therefore, there is a need to perform classifying expression on each type of knowledge. In accordance with the ontology-based expression way, the expression of each type of knowledge should include three contents, i.e. category, attribute and individual, i.e. metadata.

The present invention provides a system prototype of an intelligent regional mobile healthcare integrated data center based on metadata, characterized by comprising the following steps: collecting data from a mobile medical aid post, extracting and purifying the data, and loading same in a regional mobile healthcare integrated data center; providing the data for a data warehouse by aggregating the data; according to categories of mobile healthcare, extracting corresponding professional metadata from the data warehouse, to form a data market; mapping metadata of a knowledge base to dictionary data of a service system by matching and merging logics, to provide uniform analysis paths and data services. The system prototype of an intelligent regional mobile healthcare integrated data center based on metadata is constructed.

### SUMMARY

The present invention aims to provide a system prototype of an intelligent regional mobile healthcare integrated data center based on metadata. The present invention has the following features:

### Technical Solution of the Invention

1. A system architecture of an intelligent regional mobile healthcare integrated data center based on a metadata, characterized by comprising the following steps:

1) collecting data from a mobile medical aid post, extracting and purifying the data, and loading same in a regional mobile healthcare integrated data center;

2) providing the data for a data warehouse by aggregating the data of the regional mobile healthcare integrated data center;

3) according to categories of mobile healthcare, extracting corresponding professional metadata from the data warehouse, to form a data market; and

4) mapping metadata of a knowledge base to dictionary data of a service system by matching and merging logics, to provide uniform analysis paths and data services.

2. Based on the architecture of claim 1, a system prototype of an intelligent regional mobile healthcare integrated data center based on metadata is constructed.

### DESCRIPTION OF DRAWINGS

FIG. 1 is a diagram of a system prototype of an intelligent regional mobile healthcare integrated data center based on metadata.

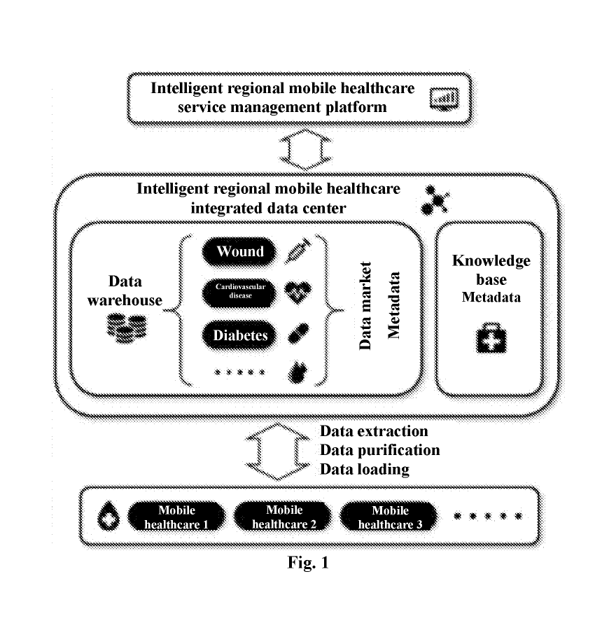

### DETAILED DESCRIPTION

Such system prototype of an intelligent regional mobile healthcare integrated data center based on metadata, characterized by comprising the following steps:

1) collecting data from a mobile medical aid post, extracting and purifying the data, and loading same in a regional mobile healthcare integrated data center;

2) providing the data for a data warehouse by aggregating the data of the regional mobile healthcare integrated data center;

3) according to categories of mobile healthcare, extracting corresponding professional metadata from the data warehouse, to form a data market;

4) mapping metadata of a knowledge base to dictionary data of a service system by matching and merging logics, to provide uniform analysis paths and data services; and

5) constructing the system prototype of an intelligent regional mobile healthcare integrated data center based on metadata.

1. A system architecture of an intelligent regional mobile healthcare integrated data center based on a metadata, characterized by comprising the following steps:
1) collecting data from a mobile medical aid post, extracting and purifying the data, and loading same in a regional mobile healthcare integrated data center;
2) providing the data for a data warehouse by aggregating the data of the regional mobile healthcare integrated data center;
3) according to categories of mobile healthcare, extracting corresponding professional metadata from the data warehouse, to form a data market; and
4) mapping metadata of a knowledge base to dictionary data of a service system by matching and merging logics, to provide uniform analysis paths and data services;

2. Based on the architecture of claim 1, a system prototype of an intelligent regional mobile healthcare integrated data center based on metadata is constructed.
